(19) United States
(12) Patent Application Publication
(10) Pub. No.: US 2015/0352071 A1
(43) Pub. Date: Dec. 10, 2015

(54) METHODS FOR REDUCING MUSCLE SORENESS AFTER EXERCISE USING BETA-HYDROXY-BETA-METHYLBUTYRATE

(71) Applicant: ABBOTT LABORATORIES, Abbott Park, IL (US)

(72) Inventors: DEBORAH STROZIER, Westerville, OH (US); STEVEN HERTZLER, Worthington, OH (US); MONICA TORTORICE, Delaware, OH (US)

(73) Assignee: ABBOTT LABORATORIES, Abbott Park, IL (US)

(21) Appl. No.: 14/735,785

(22) Filed: Jun. 10, 2015

Related U.S. Application Data
(60) Provisional application No. 62/002,573, filed on May 23, 2014.

(51) Int. Cl. A61K 31/22; A61K 38/17; A61K 38/16; A61K 31/70

(52) U.S. Cl. CPC A61K 31/22; A61K 31/70; A61K 38/1709; A61K 38/168

(57) ABSTRACT

Nutritional compositions containing beta-hydroxy-beta-methylbutyrate (HMB) are administered to an individual to reduce muscle soreness after exercise. The compositions contain a source of HMB; a protein source with at least about 21% branched-chain amino acids, by weight of total protein in the protein source; and a carbohydrate blend which contains a combination of rapid release and sustained release carbohydrates.

### CROSS-REFERENCE TO RELATED APPLICATIONS

This application claims priority to and any benefit of U.S. Provisional Application No. 62/002,573, filed May 23, 2014, the contents of which are incorporated herein by reference in their entirety.

### FIELD OF THE DISCLOSURE

The present disclosure relates to methods for reducing muscle soreness after exercise by administering nutritional compositions containing beta-hydroxy-beta-methylbutyrate (HMB) to an individual.

### BACKGROUND OF THE DISCLOSURE

Exercise can produce many health benefits. Individuals may be reluctant or unable to exercise, especially for extended periods of time, due to soreness that often results from exercise. In particular, individuals may be reluctant to exercise due to delayed onset muscle soreness (DOMS) that does not occur for a period of time after exercise (e.g., 24-48 hours). Methods and treatments that decrease DOMS may, therefore, improve the desire to exercise and the beneficial effects that individuals derive from such exercise.

### SUMMARY OF THE DISCLOSURE

Disclosed herein are methods of administering compositions containing beta-hydroxy-beta-methylbutyrate (HMB) to an individual to reduce muscle soreness after exercise. The compositions contain a source of calcium HMB; a protein source with at least about 25% branched-chain amino acids, by weight of total protein in the protein source; and a carbohydrate blend which contains a combination of rapid release and sustained release carbohydrates.

### BRIEF DESCRIPTION OF THE FIGURES

FIG. 1 is a graph showing interleukin-6 (IL-6) levels, a measure of inflammation, 24 and 48 hours after exercise under the treatments described in Example 1.

FIG. 2 is a graph showing creatine kinase levels, a measure of muscle damage, 24 and 48 hours after exercise under the treatments described in Example 1.

FIG. 3 is a graph showing counter movement vertical jump (CMVJ), a measure of endurance, 24 and 48 hours after exercise under the treatments described in Example 1.

FIG. 4 is a graph showing the pain scale, a measurement of pain felt by the subjects, 24 and 48 hours after exercise under the treatments described in Example 1.

FIG. 5 is a graph showing the sleep quality, a measure of sleep, 24 and 48 hours after exercise under the treatments described in Example 1.

### DETAILED DESCRIPTION OF THE DISCLOSURE

The methods of the present disclosure involve administering compositions containing beta-hydroxy-beta-methylbutyrate (HMB) to an individual to decrease muscle soreness after exercise. The essential features of the methods, as well as some of the many optional variations and additions, are described in detail hereafter.

The term “calcium HMB” as used herein, unless otherwise specified, refers to the calcium salt of beta-hydroxy-beta-methylbutyrate (also referred to as beta-hydroxyl-3-methyl butyric acid, beta-hydroxy isovaleric acid, or HMB), which is most typically in a monohydrate form. All weights, percentages, and concentrations as used herein to characterize calcium HMB are based on the weight of calcium HMB monohydrate, unless otherwise specified.

The term “nutritional composition” as used herein, unless otherwise specified, refers to nutritional liquids and nutritional powders, the latter of which may be reconstituted to form a nutritional liquid, and are suitable for oral consumption by a human.

The term “rapid release carbohydrate” as used herein, unless otherwise specified, refers to carbohydrates with a high glycemic index, i.e., carbohydrates with a glycemic index of 56 or higher.

The term “sustained release carbohydrate” as used herein, unless otherwise specified, refers to carbohydrates with a low glycemic index, i.e., carbohydrates with a glycemic index up to 55.

All percentages, parts and ratios as used herein, are by weight of the total product, unless otherwise specified. All such weights as they pertain to listed ingredients are based on the active level and, therefore, do not include solvents or by-products that may be included in commercially available materials, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various embodiments of the nutritional compositions used in the methods of the present disclosure may also be substantially free of any optional or selected essential ingredient or feature described herein, provided that the remaining composition still contains all of the required ingredients or features as described herein. In this context, and unless otherwise specified, the term “substantially free” means that the selected product contains less than a functional amount of the optional ingredient, typically less than about 1%, including less than about 0.5%, including less than about 0.1%, and also including zero percent, by weight of such optional or selected essential ingredient.

The nutritional compositions and methods may comprise, consist of, or consist essentially of the essential elements of the products as described herein, as well as any additional or optional element described herein or otherwise useful in nutritional composition applications.

### Product Form

The nutritional compositions including HMB, which are useful in the methods of the present disclosure, may be formulated in any known or otherwise suitable product form for oral or parenteral administration. Oral product forms are generally preferred and include any solid, liquid, or powder formulation suitable for use herein, provided that such a formulation allows for safe and effective oral delivery of the essential and other selected ingredients from the selected product form.

Non-limiting examples of solid nutritional composition forms suitable for use in the methods herein include snack and meal replacement products, including those formulated as bars; sticks; cookies, breads, cakes, or other baked goods; frozen liquids; candy; breakfast cereals; powders, granulated solids, or other particulates; snack chips or bites; frozen or retorted entrees; and so forth.

Non-limiting examples of liquid product forms suitable for use herein include snack and meal replacement products, hot or cold beverages, carbonated or non carbonated beverages, juices or other acidified beverages, milk or soy-based beverages, shakes, coffees, teas, enteral feeding compositions, and so forth. These liquid compositions are most typically formulated as suspensions or emulsions, but can also be formulated in any other suitable forms such as clear liquids, substantially clear liquids, solutions, liquid gels, and so forth.

Other non-limiting examples of suitable oral product forms include semi-solid or semi-liquid compositions (e.g., puddings, gels), as well as more conventional product forms such as capsules, tablets, caplets, pills, and so forth.

The quantity of the nutritional composition for providing an effective amount of HMB to the targeted user may be contained in one or a plurality of individual dosage forms that may be administered in single or multiple dosages per day.

The nutritional compositions contain calcium HMB as an exemplary source of HMB, which means that the compositions are either formulated with the addition of calcium HMB or are otherwise prepared so as to contain calcium HMB in the finished product. Calcium HMB is most typically added as such to the nutritional compositions during formulation. Calcium HMB monohydrate is preferred and is commercially available from Technical Sourcing International (TSI) of Salt Lake City, Utah and from Lonza Group Ltd. (Basel, Switzerland).

When the nutritional composition is a liquid, the concentration of HMB in the liquid may range up to about 10%, including from about 0.01% to about 10%, and also including from about 0.1% to about 5.0%, and also including from about 0.3% to about 2%, and also including from about 0.4% to about 1.5%, and also including from about 0.3% to about 0.6%, by weight of the nutritional liquid.

When the nutritional composition is a solid (e.g., a nutritional powder), the concentration of HMB in the solid may range up to about 10%, including from about 0.1% to about 8%, and also including from about 0.2% to about 5.0%, and also including from about 0.3% to about 3%, and also including from about 0.3% to about 1.5%, and also including from about 0.3% to about 0.6%, by weight of the nutritional powder.

The nutritional compositions may provide from about 0.1 to about 10 grams/day of HMB. Accordingly, the nutritional compositions may provide from about 0.5 to about 2.5 grams, including from about 1.0 to about 1.7 grams, including about 1.5 grams of HMB per serving, wherein a serving may be about 240 ml of ready to feed nutritional liquid or about 240 ml of reconstituted nutritional powder. An individual may be administered one serving per day, two servings per day, three servings per day, or four or more servings per day to receive the desired amount of HMB from the nutritional composition.

### Macronutrients

The nutritional compositions also include a protein source. The protein source includes about 21% branched-chain amino acids by weight of total protein in the protein source. Branched-chain amino acids are known in the art and include the amino acids leucine, valine, and isoleucine. The protein source can be any protein source known in the art that can be used in a nutritional composition as disclosed herein and can be formulated to contain the amount of branched-chain amino acids disclosed herein. Exemplary, non-limiting protein sources include hydrolyzed, partially hydrolyzed or non-hydrolyzed proteins or protein sources, and can be derived from any known or otherwise suitable nutritionally complete source such as milk (e.g., casein, whey), animal (e.g., meat, poultry, fish, egg albumen), soy, or combinations of the above proteins with incomplete proteins such as pea, rice, corn, potato, and the like. In particular embodiments, the protein source includes one or more of whey protein concentrate, milk protein concentrate, and soy protein isolate. Protein sources may be used in any combination in the nutritional composition so long as the amount of total branched-chain amino acids is approximately 21% or more of the total protein in the combined protein sources. It is generally preferred that, where present, whey protein comprises the majority of the protein source. Thus, in particular embodiments, at least about 25% of the total protein in the nutritional composition is from whey protein. In some embodiments, 100% of the total protein is from whey protein.

The nutritional compositions also include a carbohydrate blend. The carbohydrate blend includes a combination of a rapid release and a sustained release carbohydrate. Exemplary, non-limiting rapid release carbohydrates include maltodextrin, dextrose, sucrose, maltose, and glucose. Exemplary non-limiting sustained release carbohydrates include isomaltulose and waxy maize.

The nutritional compositions can contain protein and the carbohydrate blend in any combination that produces a decrease in muscle soreness after exercise. In particular embodiments, the nutritional composition contains total protein from the protein source(s) and carbohydrates in a weight ratio of between 10:1 and 1:10, including between 5:1 and 1:5, including between 2:1 and 1:2. In a preferred embodiment, the total protein and carbohydrates are present in the nutritional composition in a weight ratio of 1:2.

The combination of protein and carbohydrates can be used in combination with any effective amount of HMB, including any amount of HMB disclosed herein. Thus, in some embodiments, the nutritional compositions contain HMB, total protein, and carbohydrates in percentages of 1-10%, 20-40%, and 50-70%, by weight of the nutritional composition. In one exemplary embodiment, the nutritional composition contains HMB, total protein, and carbohydrates in an amount of about 2% HMB, about 29% total protein, and about 57% carbohydrate, by weight of the nutritional composition.

### Optional Ingredients

The nutritional compositions containing HMB, and optionally the macronutrients, may also contain other optional ingredients that may modify the physical, nutritional, chemical, hedonic, or processing characteristics of the products or serve as pharmaceutical or additional nutritional components when used in a targeted population. Many such optional ingredients are known or otherwise suitable for use in nutritional compositions and may be used in the nutritional compositions described herein, provided that such optional ingredients are safe and effective for oral administration and are compatible with the essential and other ingredients in the selected product form.

Non-limiting examples of such optional ingredients include preservatives, antioxidants, emulsifying agents, buffers, fructooligosaccharides, pharmaceutical actives, additional nutrients as described herein, colorants, flavors, thickening agents and stabilizers, and so forth.

The nutritional compositions may further comprise vitamins or related nutrients, non-limiting examples of which include vitamin A, vitamin D, vitamin E, vitamin K, thiamine, riboflavin, pyridoxine, vitamin B12, carotenoids, niacin, folic acid, pantothenic acid, biotin, vitamin C, choline, inositol, salts, and derivatives thereof, and combinations thereof.

The nutritional compositions may further comprise additional minerals, non-limiting examples of which include phosphorus, magnesium, calcium, sodium, potassium, molybdenum, chromium, selenium, chloride, and combinations thereof.

In particular embodiments, the nutritional compositions include one or more flavoring or masking agents. Suitable flavoring or masking agents include natural and artificial sweeteners, sodium sources such as sodium chloride, and hydrocolloids, such as guar gum, xanthan gum, carrageenan, gellan gum, gum acacia and combinations thereof. Such flavoring can help mask the tastes and/or textures associated with HMB and/or the other components of the nutritional composition, including the protein components such as whey protein. The flavoring used can be any flavoring that makes the nutritional composition more palatable to a user so long as the flavoring does not interfere with the ability of the nutritional composition to reduce muscle soreness after exercise. In particular embodiments, the flavoring includes one or more of a chocolate or vanilla flavoring. Particularly acceptable flavorings that provide effective masking of the off-flavors associated with the other components of the nutritional composition can be identified by hedonic testing of the nutritional composition by individuals as described in further detail in Example 2. Acceptable flavors can be identified by asking participants to score each flavor, e.g., on a scale of 1-9 (with 1 being most disliked and 9 being the most liked). In particular embodiments, acceptable flavors are those with hedonic scores at or above 4 on a scale of 1-9, including flavors with scores between 4 and 5, including those with scores between 4 and 4.5, including those with scores about 4.1, about 4.2, about 4.3, and about 4.4.

### Methods of Manufacture

The nutritional compositions may be manufactured by any known or otherwise suitable method for making nutritional compositions including nutritional liquids such as emulsions.

In one suitable manufacturing process, a nutritional liquid is prepared using at least three separate slurries, including a protein-in-fat (PIF) slurry, a carbohydrate-mineral (CHO-MIN) slurry, and a protein-in-water (PIW) slurry. The PIF slurry is formed by heating and mixing the selected oils (e.g., canola oil, corn oil, fish oil, etc.) and then adding an emulsifier (e.g., lecithin), fat soluble vitamins, and a portion of the total protein (e.g., milk protein concentrate, etc.) with continued heat and agitation. The CHO-MIN slurry is formed by adding with heated agitation to water: minerals (e.g., potassium citrate, dipotassium phosphate, sodium citrate, etc.), trace and ultra trace minerals (TM/UTM premix), thickening or suspending agents (e.g., Avicel, gellan, carrageenan), and HMB. The resulting CHO-MIN slurry is held for 10 minutes with continued heat and agitation before adding additional minerals (e.g., potassium chloride, magnesium carbonate, potassium iodide, etc.) and carbohydrates (e.g., fructooligosaccharide, sucrose, corn syrup, etc.). The PIW slurry is then formed by mixing with heat and agitation the remaining protein (e.g., sodium caseinate, soy protein concentrate, etc.) into water.

The resulting slurries are then blended together with heated agitation and the pH adjusted to the desired range, typically from 6.6-7.0, after which the composition is subjected to high-temperature short-time (HTST) processing during which the composition is heat treated, emulsified and homogenized, and then allowed to cool. Water soluble vitamins and ascorbic acid are added, the pH is again adjusted to the desired range if necessary, flavors are added, and water is added to achieve the desired total solid level. The composition is then aseptically packaged to form an aseptically packaged nutritional emulsion, or the composition is added to retort stable containers and then subjected to retort sterilization to form retort sterilized nutritional emulsions.

The manufacturing processes for the nutritional powders may be carried out in ways other than those set forth herein without departing from the spirit and scope of the present disclosure. The present embodiments are, therefore, to be considered in all respects illustrative and not restrictive and that all changes and equivalents also come within the description of the present disclosure.

The nutritional solid, such as a spray dried nutritional powder or dry-mixed nutritional powder, may be prepared by any collection of known or otherwise effective techniques, suitable for making and formulating a nutritional powder.

For example, when the nutritional powder is a spray dried nutritional powder, the spray drying step may likewise include any spray drying technique that is known for or otherwise suitable for use in the production of nutritional powders. Many different spray drying methods and techniques are known for use in the nutrition field, all of which are suitable for use in the manufacture of the spray dried nutritional powders herein.

One method of preparing the spray dried nutritional powder comprises forming and homogenizing an aqueous slurry or liquid comprising HMB, protein, and carbohydrate, and then spray drying the slurry or liquid to produce a spray dried nutritional powder. The method may further comprise the step of spray drying, dry mixing, or otherwise adding additional nutritional ingredients, including any one or more of the ingredients described herein, to the spray dried nutritional powder. The methods of manufacture are formulated with calcium HMB, which is most typically formulated as calcium HMB monohydrate as the HMB source for use in the methods.

### Methods of Use

The nutritional compositions including HMB are administered orally to an individual as needed to facilitate reduced muscle soreness after exercise. The individual may be administered any amount of the nutritional composition that is effective in reducing muscle soreness after exercise. In some embodiments, the individual is administered between 1 and 100 grams of the nutritional composition, including between 50 and 100 grams, including between 50 and 75 grams, and also including between 75 and 100 grams. In particular embodiments, the individual is administered 70 grams of the nutritional composition. While the nutritional composition can be provided to the individual in any product form described above, it is preferred that the nutritional composition be provided as a liquid produced by reconstitution of a powder form of the nutritional composition (e.g., in water or milk). The individual can be administered any volume of liquid containing a sufficient amount of the nutritional composition to effectively reduce muscle soreness after exercise. In some embodiments, the individual is administered between 4 and 16 ounces of liquid, including between 6 and 12 ounces of liquid, including 8 ounces of liquid. In a particularly specific embodiment, the individual is administered 8 ounces of liquid containing 70 grams of a powdered nutritional composition.

The nutritional composition can be administered at any time during, prior to, or after exercise and at any frequency so long as at least one of the doses is taken within 30 minutes following exercise. In some embodiments, the nutritional composition is administered between 1 and 4 times a day, including 2 and 3 times a day to the individual. In some embodiments, the nutritional composition is administered at a combination of times during, prior to, and/or after exercise. In one exemplary embodiment, one dose of the nutritional composition is administered to the individual immediately after exercise and another does is administered at some other time during the day. The nutritional composition may be administered to the individual for any period of time prior to, during, and/or after exercise, including for any number of hours, days, weeks, or months. In one exemplary embodiment, the nutritional composition is administered to the individual for one, two, three, or four weeks prior to exercise.

Individuals with decreased muscle soreness after exercise may be identified as having one or more properties suggestive of decreased muscle soreness. In some embodiments, individuals with decreased muscle soreness after exercise have one or more of reduced inflammation, reduced muscle damage, increased retention of jumping power, reduced pain, and improved sleep after exercise. In some embodiments, individuals with decreased muscle soreness have muscle soreness decreased by about 15% to about 30% including about 20% to about 30%, and, in particular embodiments, by about 28%.

### EXAMPLES

The following examples illustrate specific embodiments and/or features of the present disclosure. The examples are given solely for the purpose of illustration and are not to be construed as limitations of the present disclosure, as many variations thereof are possible without departing from the spirit and scope of the disclosure. All exemplified amounts are weight percentages based upon the total weight of the product, unless otherwise specified.

The exemplified products are HMB-containing nutritional compositions prepared in accordance with manufacturing methods well known in the nutrition industry for preparing nutritional emulsions and powders and suitable for use in the methods of the present disclosure.

### Example 1

### Individuals Administered Nutritional Compositions with HMB, Protein, and the Carbohydrate Blend (Recovery Protein) Had Reduced Muscle Soreness Relative to Individuals Who were Administered Whey Protein

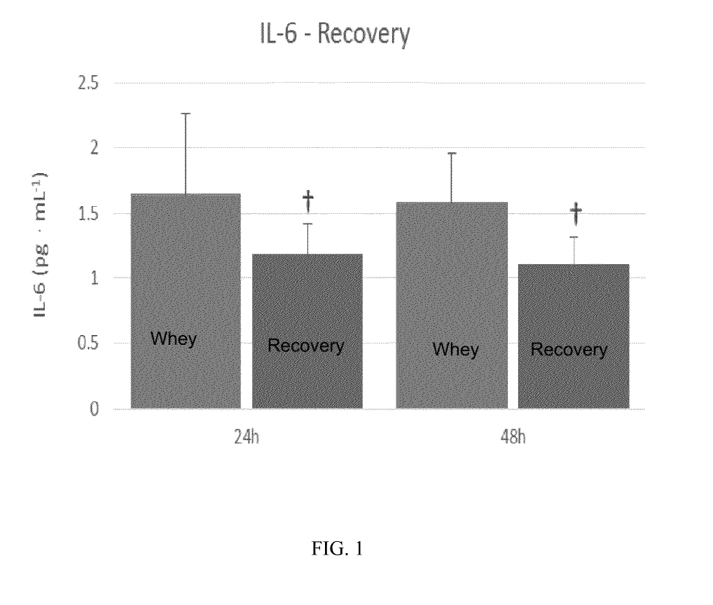

Twelve highly resistance-trained young males were recruited for the study. Baseline measurements for interleukin-6 (IL-6), creatine kinase, counter movement vertical jump (CMVJ), pain, and sleep quality were taken at the beginning of the study. Following baseline measurements, the individuals were administered an amount of either whey protein or the recovery protein composition for a two-week period such that all subjects received 20 grams of protein twice a day, with one serving consumed within 30 minutes after exercise. During the two week period, all subjects continued regular exercise. Following the two week period, all subjects underwent an intensive three day exercise regimen which included barbell squats, bench presses, dead lifts, shoulder presses, bent over rows, and lateral pull down exercises. Administration of the compositions continued during this period. Subjects were then given a two-day recovery period during which further measurements were taken while continuing administration of the compositions. Following the recovery period, the procedure was repeated with the subjects receiving the alternative composition (e.g., subjects who initially received the whey protein were switched to the recovery protein). Results of the measurements taken during the two-day recovery periods are shown in FIGS. 1-5.

As shown in FIG. 1, IL-6 levels at 24 and 48 hours after the exercise regimen during recovery protein administration were significantly lower (†) than the same period during whey protein administration, suggestive of decreased inflammation after exercise. IL-6 levels can be measured using any suitable method, for example, enzyme-linked immunosorbent assay.

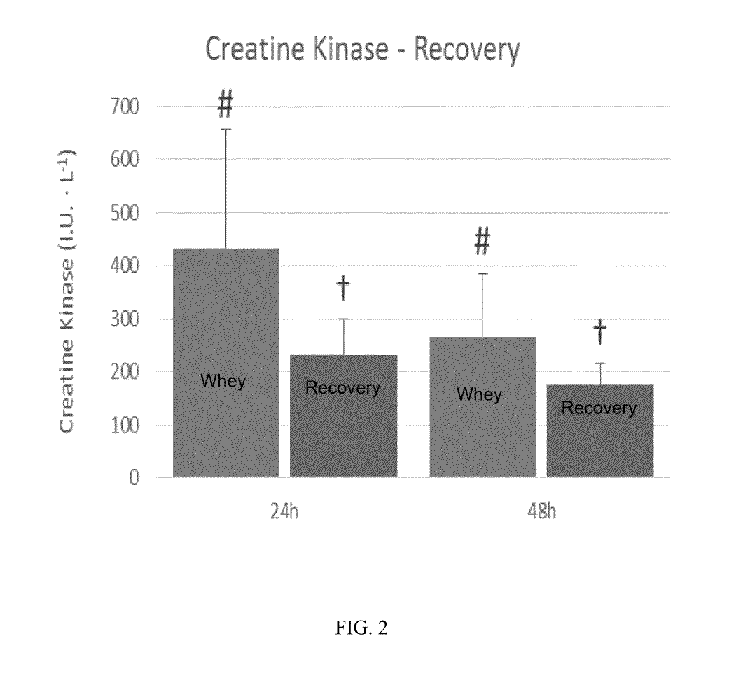

As shown in FIG. 2, creatine kinase levels at 24 and 48 hours after the exercise regimen during recovery protein administration were significantly lower (†) than the same period during whey protein administration, suggestive of decreased muscle damage after exercise. Creatine kinase levels can be measured using any suitable method, for example, a spectrophotometric method.

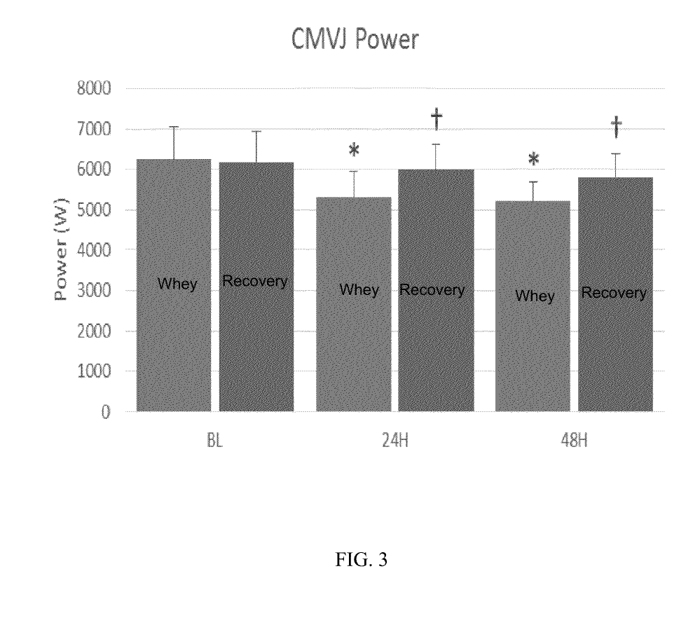

As shown in FIG. 3, CMVJ power at 24 and 48 hours after the exercise regimen during recovery protein administration were significantly higher (†) than the same period during whey protein administration, suggestive of increased endurance after exercise. Surprisingly, CMVJ power was significantly lower (*) than baseline (BL) levels during whey protein administration, but not during recovery protein administration, suggestive of better maintenance of CMVJ power even after strenuous exercise.

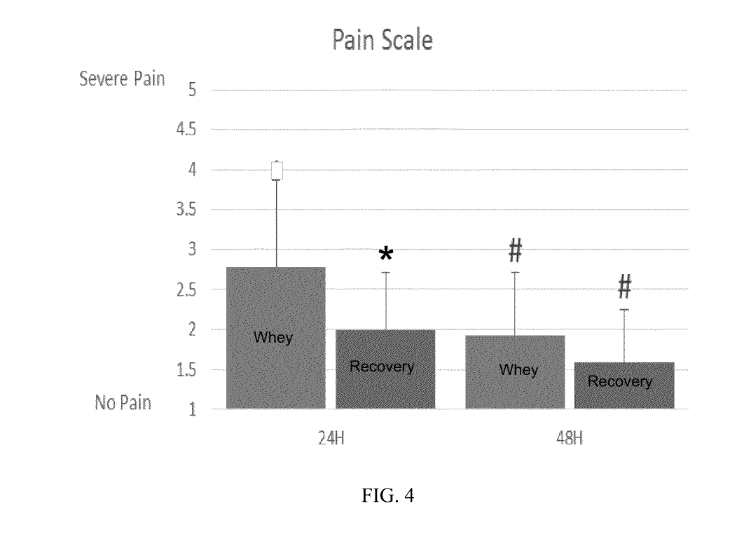

As shown in FIG. 4, the subjects' reported pain at 24 hours after the exercise regimen during recovery protein administration was significantly lower (*) than the same period during whey protein administration, suggestive of decreased muscle pain after exercise.

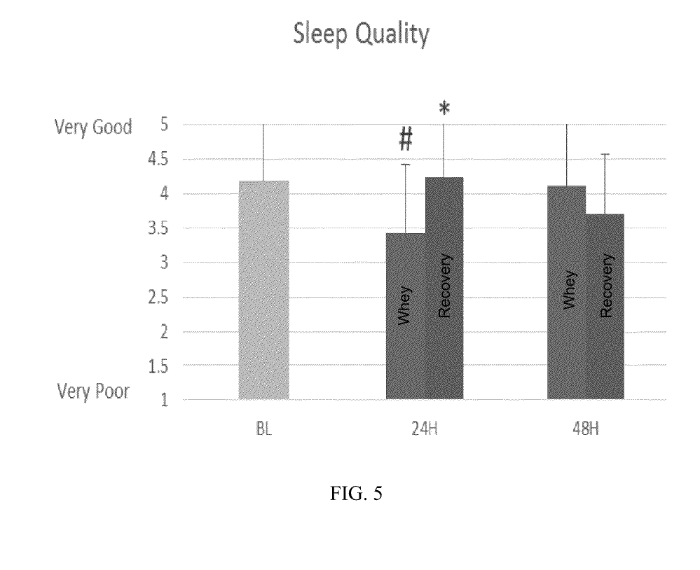

As shown in FIG. 5, the subjects' reported sleep quality at 24 hours after the exercise regimen during recovery protein administration was significantly higher (*) than the same period during whey protein administration, suggestive of improved sleep after exercise.

### Example 2

### Individuals Prefer the Taste and Feel of Nutritional Compositions with HMB, Protein, and the Carbohydrate Blend (Recovery Protein) to Whey Protein Compositions in a Side-by-Side Taste Test

Between 42 and 46 individuals participated in taste tests to determine the preference for chocolate and vanilla flavored recovery protein formulations relative to chocolate and vanilla flavored whey protein formulations. Panelists rated the compositions on a scale of 1-9 with a rating of 1 for extremely disliked flavors and a rating of 9 for extremely liked flavors. Samples were prepared according to product reconstitution rates. Samples were served at room temperature. Panelists were served approximately ¾ ounce of each sample. Samples were served in 5 oz. plastic cups coded with 3-digit random numbers. Standard white lighting was used. Samples were presented using a Balanced Complete Block Design of all possible orders and combinations. Analysis of Variance, Friedman Analysis of Rank, and the Fisher LSD test (when appropriate) were used to analyze the data. Significance was determined at the 95% confidence level. The chocolate (4.20) and vanilla (4.43) flavored recovery protein samples scored significantly higher on the scale than the chocolate (3.63) and vanilla (3.95) flavored whey protein samples.

Unless otherwise indicated herein, all sub-embodiments and optional embodiments are respective sub-embodiments and optional embodiments to all embodiments described herein. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions or formulations, and illustrative examples shown and described. Accordingly, departures may be made from such details without departing from the spirit or scope of the applicant's general disclosure herein.

What is claimed is:

1. A method of reducing muscle soreness after exercise, the method comprising administering to an individual a nutritional composition comprising:
a) a source of HMB;
b) a protein source with at least about 21% branched-chain amino acids, by weight of total protein in the protein source; and
c) a carbohydrate blend, said carbohydrate blend comprising a combination of rapid release and sustained release carbohydrates.

2. The method of claim 1, wherein the protein source comprises whey protein concentrate.

3. The method of claim 2, wherein the protein source further comprises one or more of milk protein concentrate, soy protein isolate, and pea protein concentrate.

4. The method of claim 1, wherein the nutritional composition comprises the total protein and the carbohydrates in a weight ratio of about 1:2.

5. The method of claim 4, wherein the nutritional composition comprises about 2% of the source of HMB, about 57% carbohydrate, and about 29% total protein, by total weight of the nutritional composition.

6. The method of claim 1, wherein the rapid release carbohydrate comprises maltodextrin.

7. The method of claim 1, wherein the sustained release carbohydrate comprises one or more of isomaltulose and waxy maize.

8. The method of claim 2, wherein at least about 25% of the total protein is from whey protein concentrate.

9. The method of claim 2, wherein 100% of the total protein is from whey protein concentrate.

10. The method of claim 1, wherein the nutritional composition further comprises a flavoring.

11. The method of claim 10, wherein the flavoring is a chocolate or vanilla flavoring.

12. The method of claim 1, wherein the individual is administered about 70 grams of the nutritional composition at one time.

13. The method of claim 1, wherein the nutritional composition is administered to the individual twice a day.

14. The method of claim 1, wherein the nutritional composition is administered to the individual for at least one time period within 30 minutes after exercise.

15. The method of claim 1, wherein the nutritional composition is administered to the individual prior to, during, and following the exercise, with at least one serving being administered within 30 minutes after exercise.

16. The method of claim 1, wherein the nutritional composition is administered to the individual for two weeks prior to the exercise.

17. The method of claim 1, wherein administering the nutritional composition to the individual reduces the individual's inflammation after exercise.

18. The method of claim 1, wherein administering the nutritional composition to the individual reduces the individual's muscle damage after exercise.

19. The method of claim 1, wherein administering the nutritional composition to the individual improves the individual's endurance for further exercise.

20. The method of claim 1, wherein administering the nutritional composition to the individual reduces the individual's pain after exercise.

21. The method of claim 1, wherein administering the nutritional composition to the individual improves the individual's sleep after exercise.

22. The method of claim 1, wherein the nutritional composition has a hedonic score of at least about 4 on a scale of 1-9.

23. The method of claim 1, wherein the individual exercises for a period of time sufficient to render the individual susceptible to muscle soreness.

24. The method of claim 23, wherein the period of time is at least 30 minutes.
